(19) United States
(12) Patent Application Publication
(10) Pub. No.: US 2003/0085297 A1
(43) Pub. Date: May 8, 2003

(54) Aromatic packaging bag

(76) Inventor: Wen-Wu Huang, Taipei (TW)

Correspondence Address: PRO-TECHTOR INTERNATIONAL, 20775 Norada Court, Saratoga, CA, 95070-3018, US

(21) Appl. No.: 09/993,829

(22) Filed: Nov. 5, 2001

(51) Int. Cl. A61L 9/04

(52) U.S. Cl. 239/34

(57) ABSTRACT

An aromatic packaging bag includes at least two sheets of soft surface films connected along their perimeters to define a receiving space between them, and a rigid plastic frame connected to peripheral edges of the surface films. An amount of colloidal aromatic is filled in the receiving space to outward expand the surface films into a curved configuration that provides increased deformation flexibility for the packaging bag. The aromatic keeps in contact with both surface films and gradually diffuses into ambient atmosphere via micro pores on the surface films until there is only a very small amount of residual aromatic in the aromatic packaging bag. The residual aromatic is then naturally compressed and disintegrated by atmospheric pressure into non-colloidal powder.

### FIELD OF THE INVENTION

The present invention relates to an aromatic packaging bag, and more particularly to an aromatic packaging bag having two sides directly in contact with colloidal aromatic filled therein to allow a sweet smell of the aromatic to evenly diffuse into ambient air in all directions.

### BACKGROUND OF THE INVENTION

To remove bad odor from, for example, bedroom and bathroom, people would usually use some types of aromatic-smell diffusers to diffuse sweet smell through the rooms. FIG. 1 shows a conventional aromatic-smell diffuser A having an aromatic packaging bag B inserted therein. When the aromatic packaging bag B is heated in the diffuser A, a sweet smell of the aromatic filled therein diffuses into air. FIGS. 2A to 2D show changes in the conventional aromatic packaging bag B when it is heated in the diffuser A to diffuse a sweet smell. FIG. 2A shows an initial structure of the aromatic packaging bag B before it is heated to diffuse the sweet smell. As shown, the aromatic packaging bag B includes a polyethylene (PE) surface film B1 and a rigid plastic back B2 to define a receiving space between them for containing an amount of colloidal aromatic C therein. When the rigid plastic back B2 is heated in the diffuser A, the colloidal aromatic C adheres to the PE surface film B1 to gradually diffuse the sweet smell via micro pores on the PE surface film B1.

When the sweet smell of the aromatic C diffuses via the PE surface film B1 and the aromatic C gradually decreases in volume, the PE surface film B1 also gradually sinks toward the rigid plastic back B2. FIGS. 2B to 2D show different extents to which the PE surface film B1 sinks. Since the PE surface film B1 has a limited elasticity, it would eventually stop sinking while there is still a considerable amount of aromatic C remained in the receiving space. That is, there is a large amount of aromatic C remained in the receiving space and no longer in contact with the PE surface film B1 for the sweet smell to diffuse via the micro pores on the PE surface film B1, as shown in FIGS. 2C and 2D. The remained aromatic C finally sets and becomes solid powder grains in the packaging bag B without producing any sweet smell. The aromatic packaging bag B with a large amount of unused and powdered aromatic C is not economical for use and would cause problem in environmental protection when it is discarded.

FIGS. 3A to 3D shows another conventional aromatic packaging bag B′ having a PE surface film B1′ and a rigid plastic back B2′ to define a receiving space between them for containing an amount of colloidal aromatic C′ therein. As can be seen in FIG. 3A, the colloidal aromatic C′ is initially in contact with the PE surface film B1′ and an internal pressure in the aromatic packaging bag B′ is larger than an osmotic pressure of ambient air. This condition allows a sweet smell of the colloidal aromatic C′ to diffuse via the PE surface film B1′. However, the PE surface film B1′ also has a limited deformation flexibility and would finally lose contact with the gradually decreased colloidal aromatic C′. At this point, the internal pressure in the aromatic packaging bag B′ becomes smaller than the ambient atmospheric pressure and prevents the sweet smell from diffusing via the PE surface film B1′, as shown in FIG. 3B. Thus, the aromatic packaging bag B′ also has the problem of a large amount of unused aromatic C′ remained in the bag B′ to form a waste.

FIG. 3C shows the aromatic packaging bag B′ is flatly positioned on a plane surface, such as a tabletop, with the PE surface film B1′ facing upward and the rigid plastic back B2′ in contact with the tabletop. Again, the colloidal aromatic C′ is no longer in contact with the PE surface film B1′ after a period of time and the sweet smell could not diffuse via the PE surface film B1′.

FIG. 3D shows the aromatic packaging bag B′ is flatly positioned on a plane surface, such as a tabletop, with the rigid plastic back B2′ facing upward and the PE surface film B1′ in contact with the tabletop. Since the PE surface film B1′ fitly contacts with the tabletop, the sweet smell from the colloidal aromatic C′ is completely prevented from diffusing via the PE surface film B1′.

### SUMMARY OF THE INVENTION

It is therefore a primary object of the present invention to provide an improved aromatic packaging bag, both sides of which are made of soft PE surface films to provide an increased deformation flexibility for the packaging bag, so that an aromatic filled in the packaging bag keeps in contact with the soft PE surface films over a prolonged time to minimize the amount of residual aromatic in the packaging bag.

Another object of the present invention is to provide an improved aromatic packaging bag, both sides of which are made of soft PE surface films to outward expand into a curved configuration that allows positioning of the packaging bag on a plane surface without interfering with the contact of the aromatic with the PE surface films and diffusion of a sweet smell via the PE surface films.

A further object of the present invention is to provide an improved aromatic packaging bag that is compressed by the atmospheric pressure to disintegrate residual aromatic in the packaging bag into powder, preventing problems in environmental protection caused by accumulated residual aromatic in discarded aromatic packaging bags.

To achieve the above and other objects, the aromatic packaging bag of the present invention includes at least two sheets of soft PE surface films connected along their perimeters to define a receiving space between them, and a rigid plastic frame connected to peripheral edges of the surface films. An amount of colloidal aromatic is filled in the receiving space to outward expand the surface films into a curved configuration to provide increased deformation flexibility for the packaging bag. The aromatic keeps in contact with both PE surface films for the sweet smell to gradually diffuse into ambient atmosphere via micro pores on the surface films until there is only a very small amount of residual aromatic in the aromatic packaging bag. The residual aromatic is then naturally compressed and disintegrated by atmospheric pressure into non-colloidal powder having reduced volume.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a perspective view of an aromatic-smell diffuser for heating a conventional aromatic packaging bag;

FIG. 2A is a side view showing an initial structure of the conventional aromatic packaging bag of FIG. 1;

FIG. 2B shows the conventional aromatic packaging bag of FIG. 2A is primarily changed when being heated;

FIG. 2C shows the conventional aromatic packaging bag of FIG. 2B is changed further when being heated;

FIG. 2D shows the conventional aromatic packaging bag of FIG. 2C has residual aromatic therein;

FIG. 3A is a side view showing another type of the conventional aromatic packaging bag;

FIG. 3B shows the conventional aromatic packaging bag of FIG. 3A is changed when being heated;

FIG. 3C is a side view showing the conventional aromatic packaging bag of FIG. 3A is positioned on a plane surface in a first manner;

FIG. 3D is a side view showing the conventional aromatic packaging bag of FIG. 3A is positioned on a plane surface in a second manner;

FIG. 4 is a perspective view of an aromatic packaging bag according to a first embodiment of the present invention;

FIG. 5A is a side view showing an initial structure of the aromatic packaging bag of FIG. 4;

FIG. 5B is a side view showing the aromatic packaging bag of FIG. 5A changes when the aromatic in the bag gradually decreases due to diffusion thereof;

FIG. 5C is a side view showing the aromatic packaging bag of FIG. 5A changes further when most part of the aromatic in the bag has diffused;

FIG. 5D is a side view showing a final state of the aromatic packaging bag of FIG. 5A when external air compresses the bag to disintegrate and powder the residual aromatic in the bag;

FIG. 6 is a perspective view showing the aromatic packaging bag of FIG. 4 is sealed in an outer pouch;

FIG. 7 is a perspective view of an aromatic packaging bag according to a second embodiment of the present invention;

FIG. 8 is a perspective view of an aromatic packaging bag according to a third embodiment of the present invention; and

FIG. 9 is a perspective view of an aromatic packaging bag according to a fourth embodiment of the present invention.

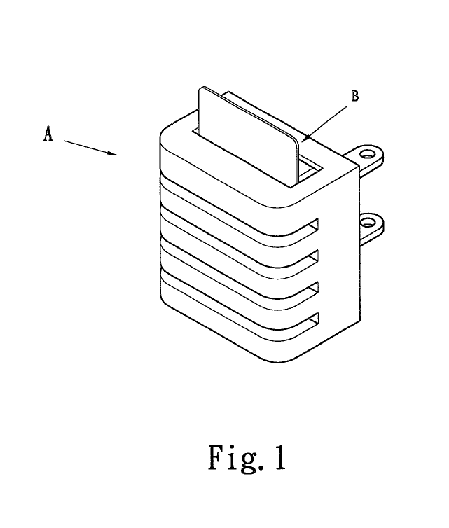

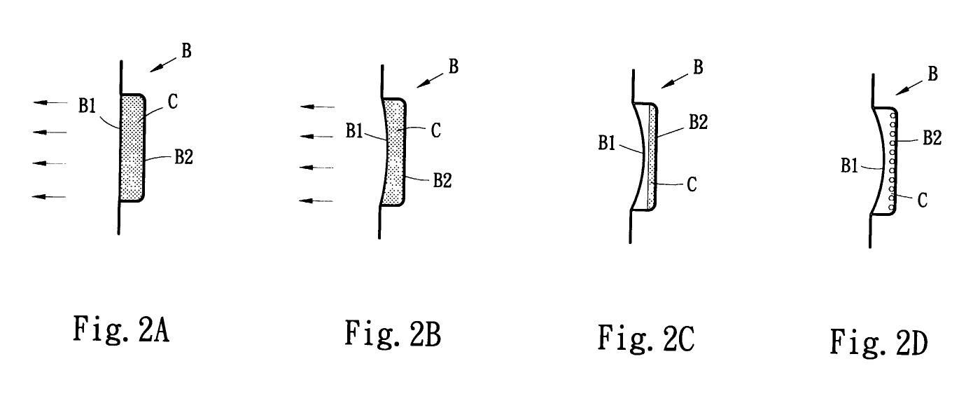

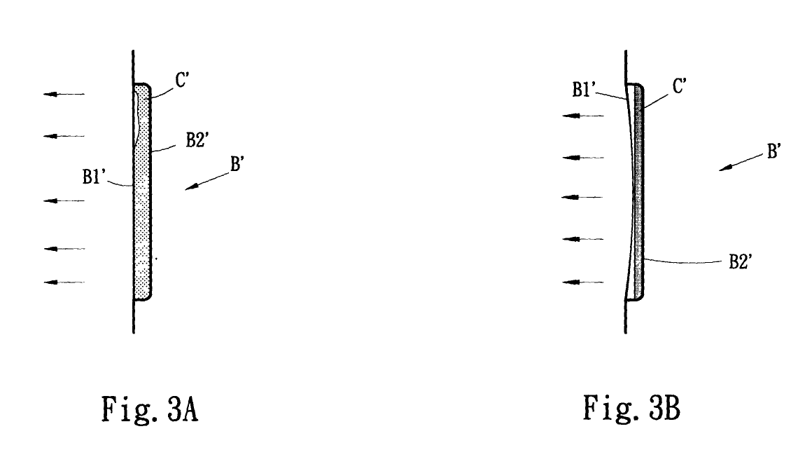

### DETAILED DESCRIPTION OF THE PREFERRED EMBODIMENTS

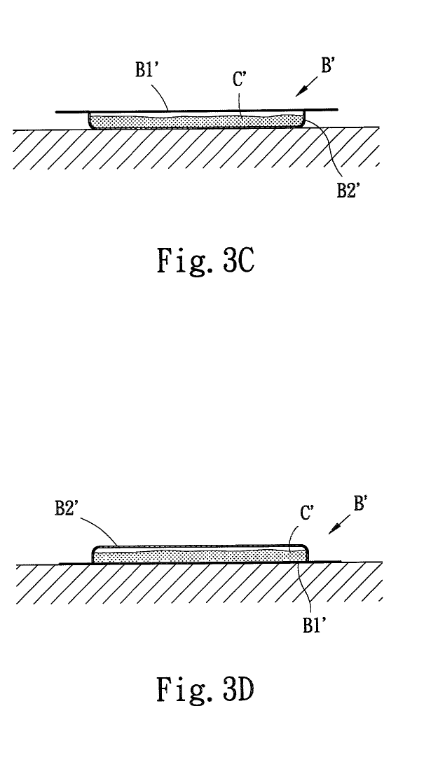

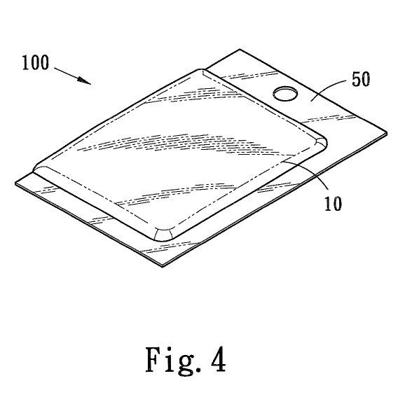

Please refer to FIGS. 4 and 5A that are perspective and sectioned side views, respectively, of an aromatic packaging bag 100 according to a first embodiment of the present invention. The aromatic packaging bag 100 includes at least two sheets of soft surface films 10 and 20. The following description of the first embodiment of the present invention is based on surface films 10, 20 made of a polyethylene (PE) material. The surface films 10, 20 are connected along their perimeters so as to define a receiving space 30 between them for an amount of colloidal aromatic 40 to fill therein and outward expand the surface films 10, 20 into a curved configuration, as shown in FIG. 5A. This expanded and curved configuration of the surface films 10, 20 allows the aromatic packaging bag 100 to have increased deformation flexibility. The connected surface films 10, 20 are then framed in a rigid plastic frame 50 to complete the packaging bag 100 and facilitate convenient holding or fixing of the packaging bag 100.

FIGS. 5B and 5C illustrate changes in the aromatic packaging bag 100 of the present invention when it is in actual use. Since the soft surface films 10, 20 separately form two sides of the packaging bag 100 to contact with the aromatic 40 filled in the packaging bag 100, a sweet smell of the aromatic 40 is able to diffuse via micro pores on the soft surface films 10, 20 even the packaging bag 100 is not heated. That is, when the receiving space 30 of the aromatic packaging bag 100 has an internal pressure larger than an external atmospheric pressure, aromatic smell automatically diffuses from the packaging bag 100 in all directions. When the aromatic 40 gradually diffuses and decreases in its volume, the soft surface films 10, 20 evenly and uniformly shrink and deform at the same time, as shown in FIG. 5B. It is the structure of the packaging bag 100 allowing uniform shrinkage of the two surface films 10, 20 that enables a constant contact of the aromatic 40 with the soft surface films 10, 20 and continuous diffusion of aromatic smell until there is only a very small amount of residual aromatic 40 in the receiving space 30, as shown in FIG. 5C. The residual aromatic 40 is initially in the form of very fine grains 41.

When the very small amount of residual aromatic 40 in the receiving space 30 is minimal and becomes the very fine grains 41, the fine grains 41 are compressed by external atmospheric pressure and could no longer diffuse any aromatic smell. Meanwhile, the external atmosphere would further compress the aromatic packaging bag 100 to disintegrate the very fine grains 41 into powder 42, making the aromatic packaging bag 100 a vacuum package having a largely reduced volume, as shown in FIG. 5D. The volume-reduced vacuum packaging bag 100 would not cause environmental pollution when it is discarded and is therefore environment-friendly.

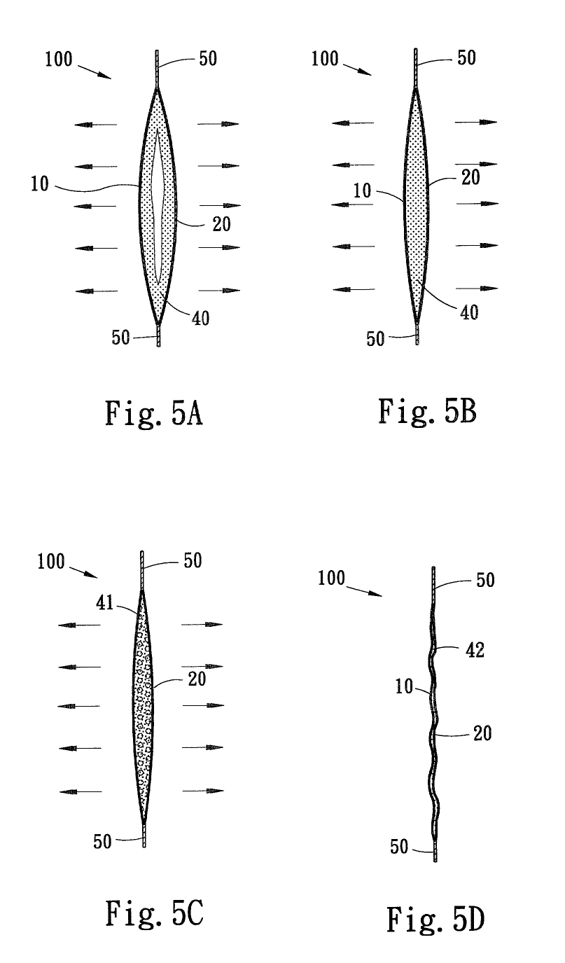

FIG. 6 shows the aromatic packaging bag 100 of the first embodiment of the present invention in an initially sealed state. More specifically, the aromatic packaging bag 100 in the first embodiment is sealed in an aluminum-foil pouch 60 before being delivered from a manufacturing plant. The pouch 60 prevents diffusion of the aromatic smell from the packaging bag 100.

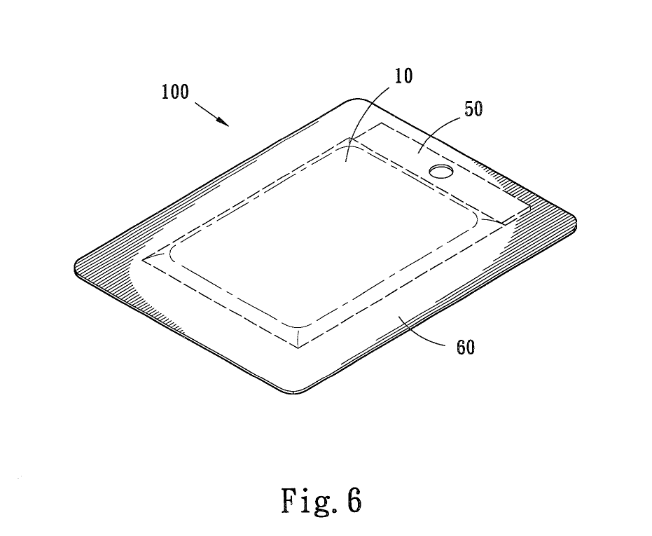

FIG. 7 shows a second embodiment of the present invention. In this embodiment, the aromatic packaging bag 100 includes a soft surface film 10 that is preformed into a closed bag for containing the aromatic 40, and the rigid plastic frame 50 is provided only at an upper edge of the packaging bag 100 for convenient hanging and holding purpose. This type of packaging bag 100 enables more effective diffusion of the sweet smell of the aromatic 40 from the packaging bag 100.

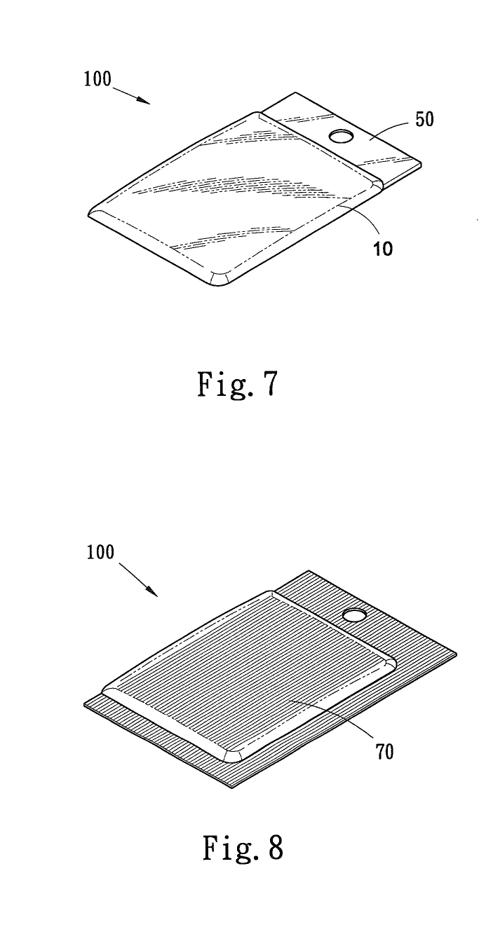

Please refer to FIG. 8 that shows a third embodiment of the present invention. The aromatic packaging bag 100 in the third embodiment is different from that in the first embodiment of FIG. 4 in that the surface films 10, 20 are directly coated at their outer surfaces with a thin layer of foil-laminated film 70 to eliminate inconveniences and additional costs due to sealing of the packaging bag 100 in the aluminum-foil pouch 60. That is, the aromatic 40 is directly received in the receiving space 30 defined between the two surface films 10, 20 coated with the foil-laminated film 70. To use the aromatic packaging bag 100 in the third embodiment, a user needs only to peel off the foil-laminated film 70 from the packaging bag 100.

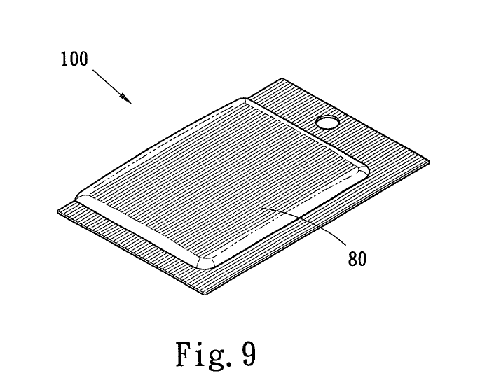

FIG. 9 shows an aromatic packaging bag 100 according to a fourth embodiment of the present invention. In this embodiment, the soft surface films 10, 20 are coated at outer surfaces with a thin layer 80 of non-woven fabric. Since plastic fibers forming the non-woven fabric have the property of adsorbing aromatic smell, the thin layer 80 of non-woven fabric enables diffusion of sweet smell from the aromatic packaging bag 100 of the present invention over an even longer time period. Like the aromatic packaging bag 100 in the first embodiment, the aromatic packaging bag 100 in the fourth embodiment of the present invention may also be sealed in an aluminum-foil pouch 60 as that in FIG. 6 before the packaging bag 100 is delivered from a manufacturing plant. To use the aromatic packaging bag 100 of the fourth embodiment, the use needs only to tear open the aluminum pouch 60.

The purpose of the aromatic packaging bags 100 of the present invention shown from FIGS. 4 to 9 is to store the aromatic 40 in a simplest possible structure, so that the aromatic packaging bags 100 provide more conveniences, economic benefits, and environmental protection values. The aromatic packaging bags 100 of the present invention are particularly practical for use due to their significant ability of largely reducing the residual aromatic in the packaging bags.

The present invention has been described with some preferred embodiments thereof illustrated in FIGS. 4 through 9, and it is understood that many changes and modifications in the described embodiment can be carried out without departing from the scope and the spirit of the invention that is intended to be limited only by the appended claims.

What is claimed is:

1. An aromatic packaging bag, comprising at least two sheets of soft surface films that are connected to each other along their perimeters to define a receiving space between them for an amount of colloidal aromatic to fill therein and outward expand said at least two sheets of surface films into a curved configuration that provides increased deformation flexibility for said packaging bag; and said aromatic being in contact with both said at least two sheets of soft surface films and gradually decreasing in volume while a sweet smell thereof diffusing into ambient atmosphere via micro pores on said surface films, causing said surface films to evenly and uniformly shrink and deform at the same time while keeping in contact with said aromatic over a prolonged time.

2. The aromatic packaging bag as claimed in claim 1, wherein said soft surface films are polyethylene (PE) films.

3. The aromatic packaging bag as claimed in claim 1, wherein said soft surface films are coated at respective outer surfaces with a layer of foil-laminated film.

4. The aromatic packaging bag as claimed in claim 1, wherein said soft surface films are coated at respective outer surfaces with a layer of non-woven fabric.

5. The aromatic packaging bag as claimed in claim 1, further comprises a rigid plastic frame connected to said perimeters of said soft surface films.

6. The aromatic packaging bag as claimed in claim 1, wherein said soft surface films together form a closed bag that is connected at a top edge to a rigid plastic sheet.

7. The aromatic packaging bag as claimed in claim 1, wherein said aromatic packaging bag is sealed in an aluminum-foil pouch.
